(19) United States
(12) Patent Application Publication
(10) Pub. No.: US 2012/0209146 A1
(43) Pub. Date: Aug. 16, 2012

(54) SOFT TISSUE ELASTICITY DISTRIBUTION MEASUREMENT METHOD AND SOFT TISSUE ELASTICITY DISTRIBUTION MEASUREMENT DEVICE

(75) Inventors: Takeo Matsumoto, Nagoya (JP); Shinichi Miyano, Tokyo (JP); Kazuaki Nagayama, Nagoya (JP)

(73) Assignee: NATIONAL UNIVERSITY CORPORATION NAGOYA INSTITUTE OF TECHNOLOGY, Nagoya-shi, Aichi (JP)

(21) Appl. No.: 13/504,718

(22) PCT Filed: Nov. 4, 2010
(86) PCT No.: PCT/JP10/69568

§ 371 Date: Apr. 27, 2012

(30) Foreign Application Priority Data
Nov. 5, 2009 (JP) 2009-254316

(51) Int. Cl. A61B 5/103

(52) U.S. Cl. 600/587

(57) ABSTRACT

An aspiration chamber is provided with an aspiration aperture, having a shape in which a width becomes larger from one edge toward another edge, and aspirates soft tissue through the aspiration aperture. A deformation amount measurement portion measures aspiration deformation amounts of the soft tissue within the aspiration aperture along a virtual line from the one edge to another edge. Based on the aspiration deformation amounts that have been measured by the deformation amount measurement portion, a computer uses a finite element model of the soft tissue to derive an approximation equation according to a numerical function for the aspiration deformation amounts and positions on the virtual line and determines a distribution of elasticity from the surface of the soft tissue into its interior by substituting parameters of the approximation equation into estimation equations that are derived by assuming that the deformation along the virtual line reflects elasticity distribution parameters.

### TECHNICAL FIELD

The present invention relates to a soft tissue elasticity distribution measurement method and a soft tissue elasticity distribution measurement device that measure the elasticity distribution of soft tissue from its surface toward its interior.

### BACKGROUND ART

Soft biological tissue can be described as a composite material that is made up of elements that have various mechanical properties. For example, human skin can be divided, starting from the surface, into the horny layer, the epidermis, and the dermis. The mechanical properties of these tissues, such as their elastic moduli and the like, differ significantly due to differences in the composition and structure of elements with different mechanical properties, including keratinocytes, melanocytes, collagen fibers, and elastin fibers. Therefore, in many cases, the elastic modulus of soft tissue varies according to the depth from the surface.

In order to gain an accurate understanding of the mechanical properties of this sort of soft biological tissue, the inventors have pursued the development of a probe-type skin elasticity measurement system. The system uses a pipette aspiration method.

The pipette aspiration method is a method in which the tip of a pipette is brought into contact with the surface of a specimen, and the elastic modulus of the specimen is estimated by measuring the amount of deformation in the surface of the specimen that occurs due to negative pressure that is applied to the interior of the pipette and comparing it to the result of an analysis by the finite element method.

Specifically, a circular tube (or a plate with a circular opening in it) is pressed lightly against the surface of the specimen, as shown in FIG. 9, and negative pressure is applied to the interior of the tube, causing the specimen to be aspirated into the tube. The elastic modulus (Young's modulus) of the specimen is determined by comparing the relationship between an aspiration pressure AP and an aspiration amount L of the specimen that is aspirated into the tube to the results of a computer simulation (an FEM analysis).

The range within which the elastic modulus is measured by this method is determined by analyzing and testing the area from the surface of the specimen to the depth of the tissue within the diameter of the pipette (refer to NPL 1, for example). Accordingly, based on the deformation behavior when the specimen is aspirated by pipettes with several different aspiration aperture diameters, a method has been proposed that posits a two-layered model that includes a top layer and a base layer and that determines the elastic modulus and the thickness of the top layer and the elastic modulus of the base layer. The effectiveness of this method has been confirmed (refer to NPL 2, for example).

### [Citation List]

### [Non-Patent Literature]

### [NPL 1]

### T. Aoki, et al., Annals of Biomedical Engineering 25, 581-587, 1997.

### [NPL 2]

### T. Matsumoto, T. Kawawa, Y. Nagano, M. Sato, Basic Study of Separate Measurements of Elasticity Characteristics of Epidermis and Dermis of Skin by Pipette Aspiration Method, 13th JSME Bioengineering Conference, 228-229 (2001).

### SUMMARY OF INVENTION

### [Technical Problem]

However, with the conventional pipette aspiration method that is used in the device that is disclosed in NPL 2, the aspirating is performed through an opening for which the cross section (the planar shape) is circular, so it is necessary to carry out the measuring by bringing pipettes (or aspiration apertures) with different diameters into contact with the specimen any number of times, which makes the measuring cumbersome.

Furthermore, in a case where the state of the measured object changes from moment to moment, as in the case of a measurement of a human subject, a problem may arise in the precision of the measurement, and the measurement requires considerable time.

In light of the foregoing, it is an object of the present invention to provide a soft tissue elasticity distribution measurement method and a soft tissue elasticity distribution measurement device that can determine the distribution in the thickness direction of elasticity of soft tissue in a simple manner.

### [Solution to Problem]

In order to achieve the above-described object, the present invention includes bringing a material into contact with a surface of a soft tissue, the material being provided with an aperture that has a shape in which a width becomes larger from one edge toward another edge, and the material restricting displacement of the soft tissue in a vertical direction; aspirating the soft tissue by applying a negative pressure from the opposite side of the aperture from the soft tissue; measuring aspiration deformation amounts of the soft tissue within the aperture along a virtual line from the one edge to said another edge; and determining a distribution in a thickness direction of elasticity of the soft tissue, based on the aspiration deformation amounts.

The distribution in the thickness direction of the elasticity of the soft tissue can thus be determined easily by a single round of measurement.

In order to achieve the above-described object, the present invention includes an aspiration chamber, a deformation amount measurement portion, and a computer. The aspiration chamber is provided with an aspiration aperture, which has a shape in which a width becomes larger from one edge toward another edge, and aspirates soft tissue through the aspiration aperture. The deformation amount measurement portion measures aspiration deformation amounts of the soft tissue within the aspiration aperture along a virtual line from the one edge to said another edge. The aspiration deformation amounts that are measured by the deformation amount measurement portion are input to the computer. The computer determines the distribution in the thickness direction of elasticity of the soft tissue based on the aspiration deformation amounts that are measured by the deformation amount measurement portion.

The distribution in the thickness direction of the elasticity of the soft tissue can thus be determined easily by a single round of measurement.

### BRIEF DESCRIPTION OF DRAWINGS

FIG. 1 is a figure that shows an overall configuration of a device according to an embodiment of the present invention.

FIG. 2 is an oblique view that shows a measurement portion in FIG. 1.

FIG. 3 is an oblique view in which a two-layered model that includes a top layer and a base layer is shown in three dimensions.

FIG. 4 is a graph that shows an example of an aspiration deformation amount along the axis of symmetry of an isosceles triangular opening.

FIG. 5 is a graph that shows an example of numeral calculation results for a ratio of the aspiration deformation amount.

FIG. 6 is a graph that shows an example of a relationship between an x coordinate x_{f} of a point of inflection in an Equation (2) and a thickness h of the top layer.

FIG. 7 is a graph that shows an example of a relationship between an elastic modulus E_{b} of the base layer and a parameter C of the Equation (2).

FIG. 8 is a flowchart that shows calculation processing by a computer.

FIG. 9 is a section view that shows a concept of a pipette aspiration method.

### DESCRIPTION OF EMBODIMENTS

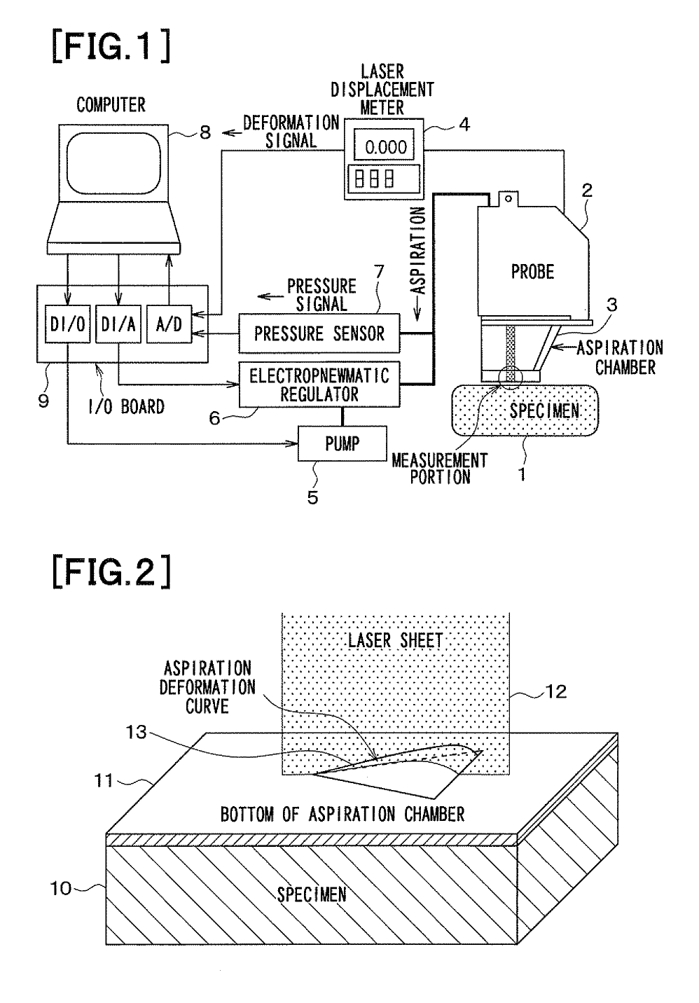

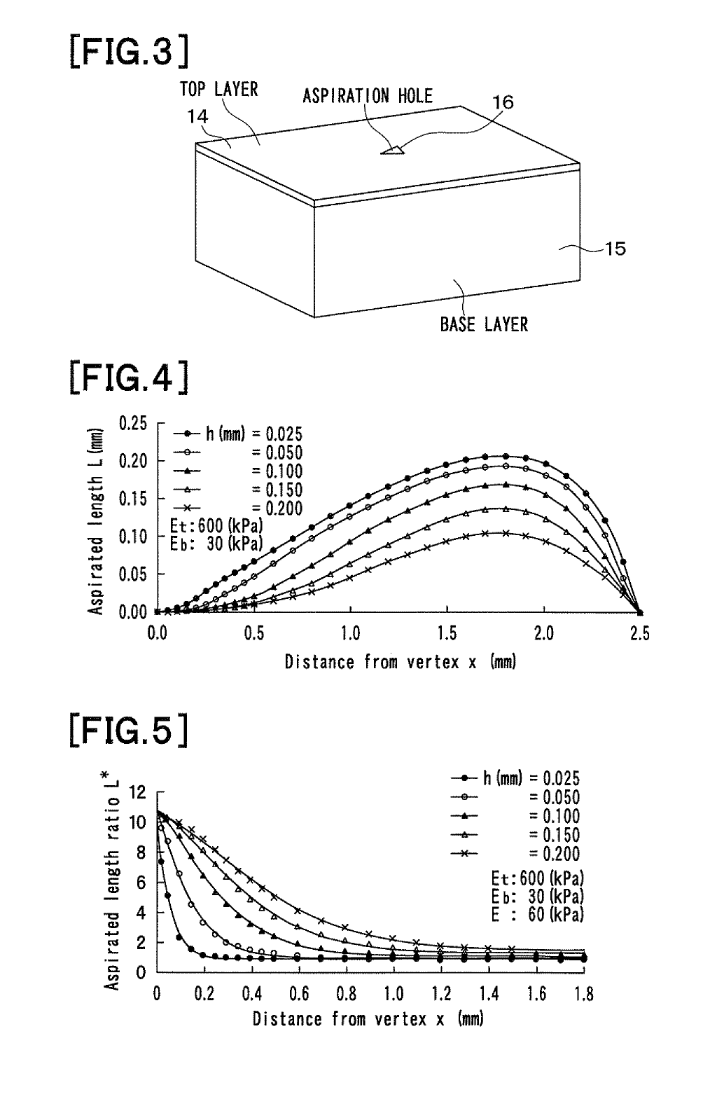

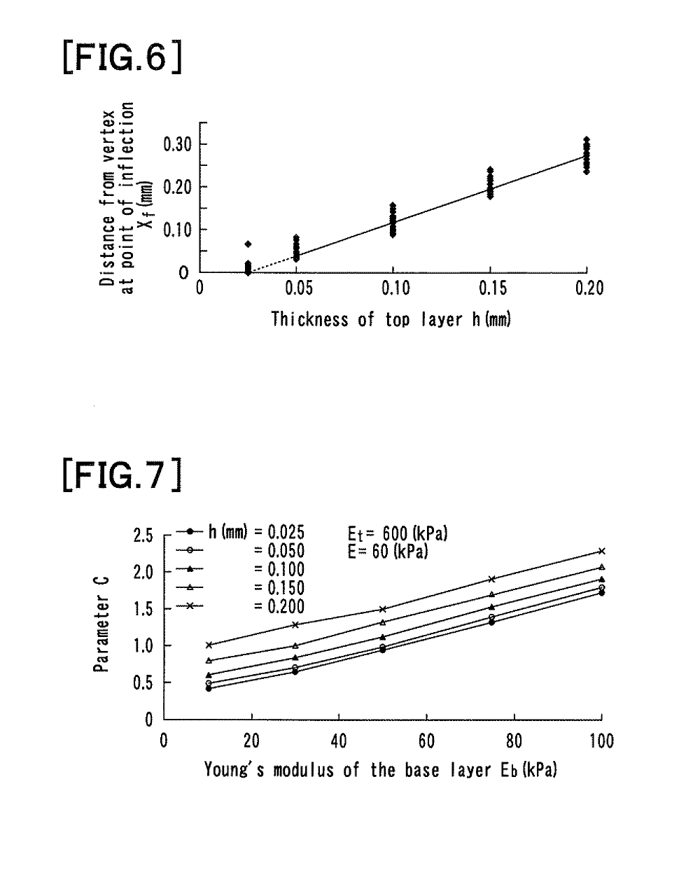

Next, an embodiment of the present invention will be described in detail with reference to FIG. 1, FIG. 2, FIG. 3, FIG. 4, FIG. 5, FIG. 6, FIG. 7, and FIG.

An example of the embodiment is shown in FIG. 1. A probe 2 is brought into contact with a specimen 1, and the specimen is aspirated into an aspiration chamber 3 in a lower portion of the probe 2 by using a pump 5 to apply a negative pressure inside the aspiration chamber 3. An aspiration pressure is controlled by an electropneumatic regulator 6 as it is measured by a pressure sensor 7 that serves as a pressure measurement portion. The measuring is controlled by a computer 8, and data transfer is performed through an I/O board 9.

An isosceles triangular opening (an aspiration aperture) is provided in a bottom side of the aspiration chamber, as shown in FIG. 2, and the deformation of the specimen along a line (a virtual line) that passes through the vertex of the opening is measured by a laser displacement meter 4 that serves as a deformation amount measurement portion. The laser displacement meter that is used here is not the widely used type that uses a laser beam to measure the displacement of a single point, but is a type that uses a laser sheet and can measure the displacement along a line that traverses the sheet. Note that it is also permissible to use a plurality of laser beams to measure the displacement at a plurality of positions along the line that passes through the vertex of the opening.

The aspiration deformation amounts along the line are determined in a state in which the specimen is subject to a constant pressure, and the Young's modulus and the thickness of a top layer are estimated by using an algorithm that is hereinafter described.

A finite element model (a mechanical model) is created in which the specimen is aspirated through an isosceles triangular opening with a base of 2 millimeters and a height of 2.5 millimeters (FIG. 3). The model, which simulates soft tissue, is a two-layer finite element model that is assumed to be non-compressible and that has a top layer and a base layer that have different elastic moduli. An aspiration pressure of 10 kPa is applied to the aspiration aperture portion.

The walls of the aperture are assumed to be rigid, the displacement of the specimen surface in the area where it is touched by the probe is restricted only in the vertical (z) direction, and other surfaces can be freely displaced. The two-layered model is defined by a top layer elastic modulus Eₜ, a base layer elastic modulus E_{b}, and a top layer thickness h, and 125 versions of the two-layered model are created by substituting the value that are shown in Table 1 for each of the parameters.

TABLE 1
Values applied to the two-layered model.

| Parameter | Values |
|---|---|
| Eₜ (kPa) | 150, 300, 600, 1500, 8000 |
| E_{b} (kPa) | 10, 30, 50, 75, 100 |
| h (mm) | 0.025, 0.050, 0.100, 0.150, 0.200 |

The distribution of the aspiration deformation amount along the axis of symmetry of the isosceles triangle is derived as a displacement L of the specimen in the z direction. The distribution is derived as a function of a distance x from the vertex. An example of an aspiration amount distribution L(x) is shown in FIG. 4.

The relationship between the distribution of the aspiration amount along the axis of symmetry, as derived by the finite element method, and the parameters Eₜ, E_{b}, and h that express the elasticity distribution in the two-layered model is examined, and an elasticity distribution parameter is estimated based on the distribution of the aspiration amount, as hereinafter explained.

(Normalization by Aspiration Amount of Single-Layered Model)

In order for the distribution of the aspiration amount along the axis of symmetry in the two-layered model to be handled uniformly under various conditions, an aspiration amount ratio L*(x) in Equation I is determined by taking an aspiration amount distribution L₀(x) in a single-layered model in which an elastic modulus E is 60 kPa and dividing it by the aspiration amount distribution L(x) in each version of the two-layered model.

L*(x)=L₀(x)/L(x) (1)

The aspiration amount ratio L*(x) expresses an approximation of the ratio of the elastic moduli that are observed at various depths in the two-layered model to the elastic modulus in the single-layered model. Note that the aspiration amount is extremely large in all of the series when x is 1.8 mm, so values in the range of 0≦x≦1.8 will be examined.

(Approximation by Numerical Function)

The distribution of the aspiration amount ratios is approximated by Equation 2, and parameters A, B, C, and n are determined such that the distribution will most closely approximate the calculation results.

L*(x)Aexp(−Bx*)+C (2)

A representative example of the approximation is shown in FIG. 5. The correlation between the approximated curve and the calculation results is 0.995 at the lowest.

(Estimation of Eₜ)

With the pipette aspiration method, the depth to which the elastic modulus can be detected is equal to the diameter of the aspiration aperture, so it is thought that in the vicinity of the vertex of the triangular opening, which is regarded as simulating a small aperture, the elastic modulus is estimated only for the tissue that is close to the surface. Converting this to the two-layered model, the elastic modulus Eₜ of the top layer is measured in the vicinity of the vertex. In other words, when the aspiration is performed with the two-layered model, which has the thin top layer and the thick base layer, what is measured in the vicinity of the vertex is the elastic modulus of the top layer. That is, when x=0 in Equation 2, the value A+C would be expected to become equal to Eₜ/E. However, a slight discrepancy is actually observed, so the accuracy of the estimate is improved by using Equation 3, which compensates for the discrepancy.

$${E}_{t} = \frac{E ( A + C )}{0.0687 {log}_{10} ( 0.016 E ( A + C ) )} \qquad ( 3 )$$

(Estimation of h)

The relationship between an x coordinate x_{f} of a point of inflection in Equation 2 and the thickness h of the top layer is shown in FIG. 6. It can be seen that, except for the case where h=0.025 millimeters, the two values have a nearly linear relationship (a correlation coefficient of 0.97). When the results of calculations with all 125 versions of the model are incorporated into FIG. 6, it can be seen that the accuracy of the estimate of h and E_{b} is low with the version in which Eₜ=3000 kPa. Accordingly, h is estimated using a straight line approximation (Equation 4) for the range in which h is 0.05 and 0.20 and Eₜ is 150 to 1500 kPa (the solid line in FIG. 6).

$$h = \frac{{x}_{f}}{1.558} + 0.0295 , where {x}_{f} = \sqrt[n]{\frac{n - 1}{nB}} \qquad ( 4 )$$

(Estimation of E_{b})

It can be seen that the base layer elastic modulus E_{b}, has an almost linear relationship to the parameter C (FIG. 7). Furthermore, C is thought to reflect the amount of aspiration in the vicinity of the center of gravity of the triangle, that is, the elastic modulus of the tissue from the surface to a certain depth, so it is predicted that the elasticity distribution can be expressed by a function in which the elasticity distribution parameters Eₜ, E_{b}, and h of the two-layered model serve as variables. Accordingly, when the form of the function is studied by examining the relationships between C and Eₜ and between C and h, it can be seen that there is an almost linear relationship between C and Eₜ (a correlation coefficient not less than 0.99). It can also be seen that the slope of the relationship between Eₜ and C is dependent on h.

Accordingly, Equation 5 is created to express the relationships described above, and coefficients p, q, and r are determined by numerical calculation. Equation 6 is an equation that solves for the value of E_{b} by substituting the coefficients that were determined by Equation 5, so E_{b} can be estimated by these equations. Note that the accuracy of the estimates is low in the model in which E_{b}=10 kPa and in the model in which Eₜ=3000 kPa, so the coefficients for the estimation Equation 6 are determined for the range in which E_{b} is 30 to 100 kPa and Eₜ is 150 to 1500 kPa.

$$\begin{matrix}C = {pE}_{b} + {qE}_{t} {h}^{r} & ( 5 ) \\ {E}_{b} = \frac{C - 0.009 {E}_{t} {h}^{1.21}}{0.016} & ( 6 )\end{matrix}$$

The aspiration deformation amounts that are determined along the line that passes through the vertex of the opening in a state in which the specimen is subject to a constant pressure are compared to the aspiration deformation amount distribution in the single-layered model, the parameters A, B, C, and n in Equation 2 are determined by numerical calculation, and the elasticity distribution parameters Eₜ, E_{b}, and h are estimated by Equations 3, 4, and 6.

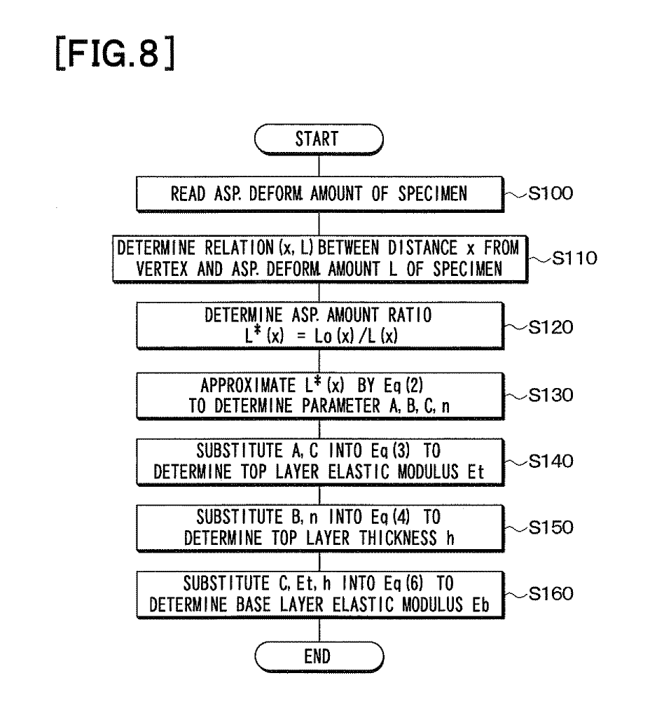

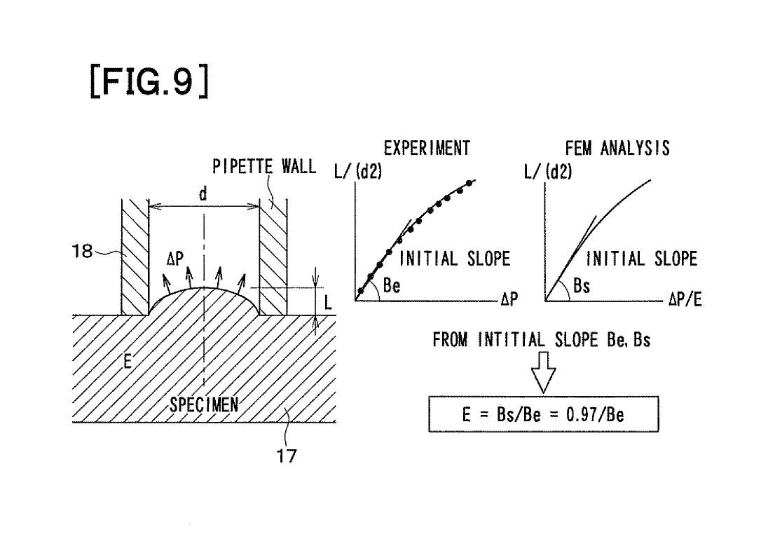

Specifically, the calculation processing that is shown by the flowchart in FIG. 8 is performed by the computer 8.

First, at Step S100, the aspiration deformation amounts for the specimen are read in the form of displacement signals from the laser displacement meter 4.

Next, at Step S110, the relationship (x, L) between the distance x from the vertex of the isosceles triangular aspiration aperture and the aspiration deformation amount L of the specimen is determined.

Next, at Step S120, the aspiration amount ratio distribution L*(x) is determined by Equation 1. Note that the aspiration amount distribution L₀(x) that is used in Equation 1, that is, the aspiration amount distribution L₀(x) in the single-layered model in which the elastic modulus E is 60 kPa, is stored in the computer 8 in advance.

Next, at Step S130, the aspiration amount ratio distribution L*(x) is approximated by Equation 2, which was described earlier, and the parameters A, B, C, and n in Equation 2 are determined. In the present example, the parameters A, B, C, and n are set such that the difference between an actual measured value Li* for L* at a distance xi from the vertex and a theoretical value Li*′=Aexp (−Bxⁿ)+C for L* at the distance xi will be minimized. To be specific, first, suitable initial values are assigned to the parameters A, B, C, and n. Then repeated calculations are made, changing the parameters A, B, C, and n a little at a time such that E in Equation 7 becomes smaller. When E ceases to become any smaller, the set of the parameters A, B, C, and n is deemed to have been solved.

$$E = {\sum }_{i} {( {L}_{i}^{*} - {L}_{i}^{* ′} )}^{2} \qquad ( 7 )$$

Next, at Step S140, the parameters A and C that were determined at Step S130 are substituted into Equation 3, and the top layer elastic modulus Eₜ is determined.

Next, at Step S150, the parameters B and n that were determined at Step S130 are substituted into Equation 4, and the top layer thickness h is determined.

Next, at Step S160, the parameter C, the top layer elastic modulus Eₜ, and the top layer thickness h, which have been determined at Steps S130 to S150, are substituted into Equation 6, and the base layer elastic modulus E_{b} is determined.

In this manner, the elasticity distribution parameters Eₜ, E_{b}, and h are determined by the computer 8.

An example of the results for the estimating of Eₜ, E_{b}, and h by the procedure described above, with the aspiration amount distribution that is determined by the finite element method being compared to the aspiration amount distribution for the single-layered model, is shown in Table 2. As shown in Table 2, it can be seen that the elasticity distribution in the two-layered model can be estimated with high accuracy by the present invention.

TABLE 2
Examples of parameter estimation. (numerical/estimation)

| Parameter | Model 1 | Model 2 | Model 3 | Model 4 |
|---|---|---|---|---|
| Eₜ (kPa) | 600/619 | 1500/1460 | 160/168 | 1500/1518 |
| E_{b} (kPa) | 50/61 | 100/100 | 80/36 | 30/18 |
| h (mm) | 0.100/0.094 | 0.200/0.203 | 0.050/0.049 | 0.150/0.168 |

It is also possible to estimate the Young's modulus distribution for multiple layers by modifying the algorithm that has been explained above.

In the embodiment that is described above, the aspiration aperture is an isosceles triangle, but the present invention is not limited to this example, and any shape that has a portion in which a width of the aperture becomes larger from one edge of the portion toward another edge of the portion may be used. For example, a diamond shape, a teardrop shape, and the like have been considered.

According to the present embodiment, the measuring in the conventional pipette aspiration method, which must be performed any number of times, because the aspirating is performed through an aperture that has a circular cross section (planar shape), is replaced by a method that enlarges the shape of the aperture into a shape that has a portion in which the width becomes larger from one edge of the portion toward another edge of the portion, creating locations where, depending on the position within the aperture, a shape is formed that is equivalent to the shape when the aspirating is performed with a small aperture and a shape is formed that is equivalent to the shape when the aspirating is performed with a large aperture, thus making it possible to determine the mechanical properties of the specimen with a single round of measurement, based on the aspiration deformation amounts within the aperture along a virtual line from the one edge to said another edge.

Furthermore, the aspiration deformation amounts along the virtual line from the one edge to said another edge are measured with precision, an equation for the relationship between the aspiration deformation amounts and the positions on the virtual line is derived, and the elastic moduli are expressed by parameters in the relationship equation in such a way that the deformation on the virtual line in the vicinity of the vertex reflects the elastic properties of only the top layer of the specimen and the deformation in the vicinity of the center of gravity of the aperture indicates the elastic properties of the deep portion of the specimen. This makes it possible to measure the distribution of the mechanical properties in the thickness direction of a soft tissue specimen with high precision, because a method is formulated for determining the distribution of the elasticity of the specimen in the depth direction, and the results of calculations by the relationship equation for the aspiration deformation amounts and their positions are utilized in order to estimate the distribution in the thickness direction of the elasticity of the specimen, as well as the thickness of the top layer, with high precision, based on the distribution of the aspiration deformation amounts within the aperture.

The laser displacement meter and the aspiration chamber are also provided. When the probe, which is characterized by using the triangular aspiration aperture in the bottom side of the aspiration chamber as its measurement portion, is brought into contact with a portion of the soft tissue specimen, a signal that indicates the pressure within the aspiration chamber, as measured by the pressure sensor that is connected to the probe, and a signal that indicates the displacement of the specimen, as measured by the laser displacement meter, are input to the computer that controls the individual measurements. A control signal that is computed by the computer such that the pressure within the aspiration chamber will remain constant is output to the electropneumatic regulator and the pump that are connected to the probe, bringing about a state in which a constant negative pressure is applied inside the aspiration chamber. Because the specimen is aspirated into the interior of the chamber through the triangular aspiration aperture, the aspiration deformation amounts along the line that passes through the vertex of the aspiration aperture in the state in which the constant pressure is applied are measured by the laser displacement meter as displacements on a line that follows the line of the deformation. Then, based on the displacements, the approximation equation is determined according to the numerical function for the positions and the displacement amounts. The parameters of the approximation equation that have been determined as just described are substituted into estimation equations that are derived by assuming that the deformation in the vicinity of the vertex on the line segment that passes through the vertex, the deformation in the vicinity of the center of gravity of the aspiration aperture, and the point of inflection of the approximation equation respectively reflect the elastic modulus Eₜ of the top layer, the elastic modulus E_{b} of the base layer, and the thickness h of the top layer. Substituting the parameters of the approximation equation into the estimation equations makes it possible to determine the distribution of the elasticity from the surface of the soft tissue into its interior, that is, the top layer elastic modulus Eₜ, the base layer elastic modulus E_{b}, and the top layer thickness h, based on a single round of measurement. Therefore, the distribution in the thickness direction of the mechanical properties of the soft tissue specimen can be measured easily and with high precision by a single round of measurement.

The mechanical properties of the soft tissue specimen are also measured by defining the aspiration aperture as an isosceles triangle and measuring the amounts of aspiration deformation of the specimen along the axis of symmetry of the triangle. This makes it possible to simplify the approximation equation for the aspiration deformation amounts along the axis of symmetry, so the mechanical properties in the thickness direction can be measured with high precision at a higher speed with a simple model such as the two-layered physical model or the like.

Furthermore, considering that, in the vicinity of the vertex of a triangular opening (aspiration aperture), only the elastic modulus close to the surface is estimated, the top layer elastic modulus Eₜ is determined by an estimation equation that is derived based on the assumption that the top layer elastic modulus is the elastic modulus when the position is close to zero in the approximation equation that describes the relationship between the aspiration deformation amount and the position with respect to the vertex. Therefore, the top layer elastic modulus Eₜ can be determined with high precision by a single round of measurement.

Moreover, the fact that the base layer elastic modulus E_{b} is determined by the estimation equation that is derived based on the fact that, in the approximation equation that pertains to the position and the aspiration deformation amount, the aspiration deformation amount in the vicinity of the center of gravity of the triangle, that is, the parameter C that reflects the elastic modulus at a certain depth from the surface, has nearly linear relationships with both the top layer elastic modulus Eₜ and the base layer elastic modulus E_{b}, as well as on the fact that the slope of the relationship between Eₜ and C is dependent on the top layer thickness h, means that the base layer elastic modulus E_{b} can be determined with high precision by a single round of measurement.

The fact that the top layer thickness h is also determined by an estimation equation that is derived based on the fact that the top layer thickness h has a linear relationship to the x coordinate of the inflection point of the approximation equation that pertains to the position and the aspiration deformation amount, means that the top layer thickness h can also be determined with high precision by a single round of measurement.

Furthermore, the fact that the estimation algorithm varies with the shape of the aspiration aperture means that the elasticity distribution can be estimated for a multi-layered model that is more complex than the two-layered physical model.

The present embodiment is characterized as hereinafter described. A material that restricts the displacement of the soft tissue in the vertical direction is provided with an aperture that has a shape in which a width becomes larger from one edge toward another edge. The material is brought into contact with the surface of the soft tissue, and the soft tissue is aspirated by applying a negative pressure from the opposite side of the aperture from the soft tissue. The aspiration deformation amounts of the soft tissue within the aperture are measured along a virtual line from the one edge to said another edge. The distribution in the thickness direction of the elasticity of the soft tissue is determined based on the aspiration deformation amounts.

The distribution in the thickness direction of the elasticity of the soft tissue can thus be determined easily by a single round of measurement.

In addition, the present embodiment is characterized in that the relationship equation for the aspiration deformation amounts and the positions on the virtual line is derived, and the distribution in the thickness direction of the elasticity is determined by using the parameters of the relationship equation to express the elastic moduli of the soft tissue.

This makes it possible to measure the distribution of the elasticity of the soft tissue in the thickness direction with high precision.

The present embodiment is also characterized in that a triangular shape is used for the shape of the aperture in the material, and the aspiration deformation amounts of the soft tissue are measured along the virtual line that passes through the vertex of the aperture.

This makes it possible to simplify the approximation equation for the aspiration deformation amounts, so the distribution in the thickness direction of the elasticity of the soft tissue can be measured at high speed.

Furthermore, the present embodiment is characterized in that the approximation equation for the aspiration deformation amounts and the positions on the virtual line is derived, and the elastic modulus Eₜ of the, top layer, the elastic modulus E_{b}, of the base layer, and the thickness h of the top layer are determined by substituting the parameters of the approximation equation into the estimation equations that have been derived by assuming that the deformation in the vicinity of the vertex, the deformation in the vicinity of the center of gravity of the aspiration aperture, and the point of inflection of the approximation equation respectively reflect the top layer elastic modulus Eₜ, the base layer elastic modulus E_{b}, and the top layer thickness h.

It is thus possible to measure the elasticity distribution in a specimen that has a top layer and a base layer with high precision and at high speed.

The present embodiment is also characterized in that the top layer elastic modulus Eₜ is determined by the estimation equation that is derived based on the assumption that the top layer elastic modulus Eₜ is the elastic modulus that is determined by the approximation equation based on the aspiration deformation behavior of the soft tissue that is estimated at a position where the distance from the vertex is zero.

The top layer elastic modulus Eₜ can thus be determined with high precision by a single round of measurement.

The present embodiment is additionally characterized in that the base layer elastic modulus E_{b} is determined by the estimation equation that is derived based on the fact that, in the approximation equation, the parameter C, which reflects the aspiration deformation amount in the vicinity of the center of gravity of the triangle, has linear relationships with both the top layer elastic modulus Eₜ and the base layer elastic modulus E_{b}, as well as on the fact that the slope of the relationship between the top layer elastic modulus Eₜ and the parameter C is dependent on the top layer thickness h.

The base layer elastic modulus E_{b} can thus be determined with high precision by a single round of measurement.

The present embodiment is further characterized in that the top layer thickness h is also determined by the estimation equation that is derived based on the fact that the top layer thickness h has a linear relationship to the x coordinate of the inflection point of the approximation equation.

The top layer thickness h can thus be determined with high precision by a single round of measurement.

The present embodiment is also characterized in that an isosceles triangular shape is used for the shape of the aperture in the material, and the aspiration deformation amounts of the soft tissue are measured along the virtual line that is coincident with the axis of symmetry of the aperture.

The approximation equation for the aspiration deformation amount can thus be further simplified, making it possible to measure the distribution in the thickness direction of the elasticity of the soft tissue at a higher speed.

Furthermore, the present embodiment is characterized in that the aspiration chamber, the deformation amount measurement portion, and the computer are provided. The aspiration chamber is provided with the aspiration aperture, which has a shape in which a width becomes larger from one edge toward another edge, and aspirates the soft tissue through the aspiration aperture. The deformation amount measurement portion measures the aspiration deformation amounts of the soft tissue within the aspiration aperture along the virtual line from the one edge to said another edge. The aspiration deformation amounts that have been measured by the deformation amount measurement portion are input to the computer. The computer determines the distribution in the thickness direction of the elasticity of the soft tissue based on the aspiration deformation amounts that have been measured by the deformation amount measurement portion.

The distribution in the thickness direction of the elasticity of the soft tissue can thus be determined easily by a single round of measurement.

The present embodiment is also characterized in that the computer determines the relationship equation for the aspiration deformation amounts and the positions on the virtual line, then determines the elasticity distribution in the thickness direction using the estimation equations that describe the elasticity of the soft tissue in terms of the parameters of the relationship equation.

The distribution in the thickness direction of the elasticity of the soft tissue can thus be measured with high precision.

The present embodiment is further characterized in that the shape of the aspiration aperture is triangular and the deformation amount measurement portion measures the aspiration deformation amounts of the soft tissue along the virtual line that passes through the vertex of the aspiration aperture.

This makes it possible to simplify the approximation equation for the aspiration deformation amounts, so the distribution in the thickness direction of the elasticity of the soft tissue can be measured at high speed.

The present embodiment is additionally characterized in that the computer derives the approximation equation for the aspiration deformation amounts and the positions on the virtual line, then determines the elastic modulus Eₜ of the top layer, the elastic modulus E_{b} of the base layer, and the thickness h of the top layer by substituting the parameters of the approximation equation into the estimation equations that are derived by assuming that the deformation in the vicinity of the vertex, the deformation in the vicinity of the center of gravity of the aspiration aperture, and the point of inflection of the approximation equation respectively reflect the top layer elastic modulus Eₜ, the base layer elastic modulus E_{b}, and the top layer thickness h.

It is thus possible to measure the elasticity distribution in a specimen that has a top layer and a base layer with high precision and at high speed.

The present embodiment is also characterized in that the computer determines the top layer elastic modulus Eₜ by using the estimation equation that is derived based on the assumption that the top layer elastic modulus Eₜ is the elastic modulus that is determined by the approximation equation based on the aspiration deformation behavior of the soft tissue that is estimated at a position where the distance from the vertex is zero.

The top layer elastic modulus Eₜ can thus be determined with high precision by a single round of measurement.

The present embodiment is further characterized in that the computer determines the base layer elastic modulus E_{b} by using the estimation equation that is derived based on the fact that, in the approximation equation, the parameter C, which reflects the aspiration deformation amount in the vicinity of the center of gravity of the triangle, has linear relationships with both the top layer elastic modulus Eₜ and the base layer elastic modulus E_{b}, as well as on the fact that the slope of the relationship between the top layer elastic modulus Eₜ and the parameter C is dependent on the top layer thickness h.

The base layer elastic modulus E_{b}, can thus be determined with high precision by a single round of measurement.

In addition, the present embodiment is characterized in that the computer determines the top layer thickness h by using the estimation equation that is derived based on the fact that the top layer thickness h has a linear relationship to the x coordinate of the inflection point of the approximation equation.

The top layer thickness h can thus be determined with high precision by a single round of measurement.

Furthermore, the present embodiment is characterized in that the shape of the aspiration aperture is isosceles triangular and the deformation amount measurement portion measures the aspiration deformation amounts of the soft tissue along the virtual line that is coincident with the axis of symmetry of the aspiration aperture.

The approximation equation for the aspiration deformation amount can thus be further simplified, making it possible to measure the distribution in the thickness direction of the elasticity of the soft tissue at a higher speed.

According to the present embodiment that is described above, it is possible to determine the elasticity distribution from the surface into the interior of a specimen of soft tissue that has a degree of softness that is comparable to that of skin and blood vessels in a single round of measurement, and to do so simply, easily, and with high precision.

Even in a case where a problem arises in the precision of the measurement, because the state of the measured object changes from moment to moment, as in the case of a measurement of a human subject, and even in a case where the measurement requires considerable time, the measurement can be completed in a single round, so precise measurements can be obtained in a short time.

Changing the shape of the aperture makes it possible to create locations where, depending on the position within the aperture, a shape is formed that is equivalent to the shape when the aspirating is performed with a small aperture and a shape is formed that is equivalent to the shape when the aspirating is performed with a large aperture, thus making it possible to determine the elasticity distribution for a multi-layered model that is more complex than the two-layered physical model, and to do so in a single round of measurement.

### REFERENCE SIGNS LIST

1 Specimen

2 Probe

3 Aspiration chamber

4 Laser displacement meter (deformation amount measurement portion)

5 Pump

6 Electropneumatic regulator

7 Pressure sensor

8 Computer

9 I/O board

10 Specimen

11 Bottom side of aspiration chamber

12 Laser sheet

13 Aspiration deformation curve

14 Top layer

15 Base layer

16 Aspiration aperture

17 Specimen

18 Pipette cross section

1. A soft tissue elasticity distribution measurement method, comprising:
bringing a material into contact with a surface of a soft tissue, the material being provided with an aperture that has a shape in which a width becomes larger from one edge toward another edge, and the material restricting displacement of the soft tissue in a vertical direction;
aspirating the soft tissue by applying a negative pressure from the opposite side of the aperture from the soft tissue;
measuring aspiration deformation amounts of the soft tissue within the aperture along a virtual line from the one edge to said another edge; and
determining a distribution in a thickness direction of elasticity of the soft tissue, based on the aspiration deformation amounts.

2. The soft tissue elasticity distribution measurement method according to claim 1,
wherein a relationship equation is derived for relationships between the aspiration deformation amounts and positions on the virtual line, and
the distribution in the thickness direction of the elasticity is determined by expressing the elasticity of the soft tissue in terms of parameters of the relationship equation.

3. The soft tissue elasticity distribution measurement method according to claim 1,
wherein a material in which the shape of the aperture is triangular is used as the material, and
the aspiration deformation amounts of the soft tissue are measured along the virtual line, which passes through a vertex of the aperture.

4. The soft tissue elasticity distribution measurement method according to claim 3,
wherein an approximation equation is derived for the aspiration deformation amounts and positions on the virtual line, and
an elastic modulus Eₜ of a top layer, an elastic modulus E_{b} of a base layer, and a thickness h of the top layer are determined by substituting parameters of the approximation equation into estimation equations that are derived by assuming that deformation in the vicinity of the vertex, deformation in the vicinity of a center of gravity of the aperture, and a point of inflection of the approximation equation respectively reflect the top layer elastic modulus Eₜ, the base layer elastic modulus E_{b}, and the top layer thickness h.

5. The soft tissue elasticity distribution measurement method according to claim 4,
wherein the top layer elastic modulus Eₜ is determined by one of the estimation equations that is derived by assuming that the top layer elastic modulus Eₜ is an elastic modulus that is determined by the approximation equation based on an aspiration deformation behavior of the soft tissue that is estimated at a position where the distance from the vertex is zero.

6. The soft tissue elasticity distribution measurement method according to claim 4,
wherein the base layer elastic modulus E_{b}, is determined by one of the estimation equations that is derived based on a fact that a parameter C reflecting the aspiration deformation amount in the vicinity of the center of gravity of the triangle bears linear relation ship with each of the top layer elastic modulus Eₜ and the base layer elastic modulus E_{b} in the approximation equation, and also based on a fact that a slope of linear relationship between the top layer elastic modulus Eₜ and the parameter C depends on the top layer thickness h.

7. The soft tissue elasticity distribution measurement method according to claim 4,
wherein the top layer thickness h is determined by one of the estimation equations that is derived based on a fact that the top layer thickness h bears linear relationship with an x coordinate of the point of inflection.

8. The soft tissue elasticity distribution measurement method according to claim 4,
wherein a material in which the shape of the aperture is isosceles triangular is used as the material, and
the aspiration deformation amounts of the soft tissue are measured along the virtual line, which is coincident with an axis of symmetry of the aperture.

9. A soft tissue elasticity distribution measurement device, comprising:
an aspiration chamber that is provided with an aspiration aperture, which has a shape in which a width becomes larger from one edge toward another edge, and that aspirates soft tissue through the aspiration aperture;
a deformation amount measurement portion that measures aspiration deformation amounts of the soft tissue within the aspiration aperture along a virtual line from the one edge to said another edge; and
a computer, into which are input the aspiration deformation amounts that are measured by the deformation amount measurement portion,
wherein the computer determines a distribution in a thickness direction of elasticity of the soft tissue, based on the aspiration deformation amounts that are measured by the deformation amount measurement portion.

10. The soft tissue elasticity distribution measurement device according to claim 9,
wherein the computer derives a relationship equation for the aspiration deformation amounts and positions on the virtual line and determines the distribution in the thickness direction of the elasticity using estimation equations that describe the elasticity of the soft tissue in terms of parameters of the relationship equation.

11. The soft tissue elasticity distribution measurement device according to claim 9,
wherein the shape of the aspiration aperture is triangular, and
the deformation amount measurement portion measures the aspiration deformation amounts of the soft tissue along the virtual line, which passes through a vertex of the aspiration aperture.

12. The soft tissue elasticity distribution measurement device according to claim 11,
wherein the computer derives an approximation equation for the aspiration deformation amounts and positions on the virtual line and determines an elastic modulus Eₜ of a top layer, an elastic modulus E_{b} of a base layer, and a thickness h of a top layer by substituting parameters of the approximation equation into estimation equations that are derived by assuming that deformation in the vicinity of the vertex, deformation in the vicinity of a center of gravity of the aspiration aperture, and a point of inflection of the approximation equation respectively reflect the top layer elastic modulus Eₜ, the base layer elastic modulus E_{b}, and the top layer thickness h.

13. The soft tissue elasticity distribution measurement device according to claim 12,
wherein the computer determines the top layer elastic modulus Eₜ by using an estimation equation, that is derived by assuming that the top layer elastic modulus Eₜ is an elastic modulus that is determined by the approximation equation based on an aspiration deformation behavior of the soft tissue that is estimated at a position where the distance from the vertex is zero.

14. The soft tissue elasticity distribution measurement device according to claim 12,
wherein the computer determines the base layer elastic modulus E_{b} by using an estimation equation that is derived based on a fact that a parameter C reflecting the aspiration deformation amount in the vicinity of the center of gravity of the triangle bears linear relationship with each of the top layer elastic modulus Eₜ and the base layer elastic modulus E_{b} in the approximation equation, and also based on a fact that a slope of linear relationship between the top layer elastic modulus Eₜ and the parameter C depends on the top layer thickness h.

15. The soft tissue elasticity distribution measurement device according to claim 12,
wherein the computer determines the top layer thickness h by using an estimation equation that is derived based on a fact that the top layer thickness h bears linear relationship with an x coordinate of the point of inflection.

16. The soft tissue elasticity distribution measurement device according to claim 12,
wherein the shape of the aspiration aperture is isosceles triangular, and
the deformation amount measurement portion measures the aspiration deformation amounts of the soft tissue along the virtual line, which is coincident with an axis of symmetry of the aspiration aperture.

17. The soft tissue elasticity distribution measurement method according to claim 5,
wherein the base layer elastic modulus E_{b}, is determined by one of the estimation equations that is derived based on a fact that a parameter C reflecting the aspiration deformation amount in the vicinity of the center of gravity of the triangle bears linear relation ship with each of the top layer elastic modulus Eₜ and the base layer elastic modulus E_{b} in the approximation equation, and also based on a fact that a slope of linear relationship between the top layer elastic modulus Eₜ and the parameter C depends on the top layer thickness h.

18. The soft tissue elasticity distribution measurement method according to claim 5,
wherein the top layer thickness h is determined by one of the estimation equations that is derived based on a fact that the top layer thickness h bears linear relationship with an x coordinate of the point of inflection.

19. The soft tissue elasticity distribution measurement method according to claim 6,
wherein the top layer thickness h is determined by one of the estimation equations that is derived based on a fact that the top layer thickness h bears linear relationship with an x coordinate of the point of inflection.

20. The soft tissue elasticity distribution measurement method according to claim 17,
wherein the top layer thickness h is determined by one of the estimation equations that is derived based on a fact that the top layer thickness h bears linear relationship with an x coordinate of the point of inflection.

21. The soft tissue elasticity distribution measurement method according to claim 5,
wherein a material in which the shape of the aperture is isosceles triangular is used as the material, and
the aspiration deformation amounts of the soft tissue are measured along the virtual line, which is coincident with an axis of symmetry of the aperture.

22. The soft tissue elasticity distribution measurement method according to claim 6,
wherein a material in which the shape of the aperture is isosceles triangular is used as the material, and
the aspiration deformation amounts of the soft tissue are measured along the virtual line, which is coincident with an axis of symmetry of the aperture.

23. The soft tissue elasticity distribution measurement method according to claim 17,
wherein a material in which the shape of the aperture is isosceles triangular is used as the material, and
the aspiration deformation amounts of the soft tissue are measured along the virtual line, which is coincident with an axis of symmetry of the aperture.

24. The soft tissue elasticity distribution measurement method according to claim 7,
wherein a material in which the shape of the aperture is isosceles triangular is used as the material, and
the aspiration deformation amounts of the soft tissue are measured along the virtual line, which is coincident with an axis of symmetry of the aperture.

25. The soft tissue elasticity distribution measurement method according to claim 18,
wherein a material in which the shape of the aperture is isosceles triangular is used as the material, and
the aspiration deformation amounts of the soft tissue are measured along the virtual line, which is coincident with an axis of symmetry of the aperture.

26. The soft tissue elasticity distribution measurement method according to claim 19,
wherein a material in which the shape of the aperture is isosceles triangular is used as the material, and
the aspiration deformation amounts of the soft tissue are measured along the virtual line, which is coincident with an axis of symmetry of the aperture.

27. The soft tissue elasticity distribution measurement method according to claim 20,
wherein a material in which the shape of the aperture is isosceles triangular is used as the material, and
the aspiration deformation amounts of the soft tissue are measured along the virtual line, which is coincident with an axis of symmetry of the aperture.

28. The soft tissue elasticity distribution measurement device according to claim 13,
wherein the computer determines the base layer elastic modulus E_{b} by using an estimation equation that is derived based on a fact that a parameter C reflecting the aspiration deformation amount in the vicinity of the center of gravity of the triangle bears linear relationship with each of the top layer elastic modulus Eₜ and the base layer elastic modulus E_{b} in the approximation equation, and also based on a fact that a slope of linear relationship between the top layer elastic modulus Eₜ and the parameter C depends on the top layer thickness h.

29. The soft tissue elasticity distribution measurement device according to claim 13,
wherein the computer determines the top layer thickness h by using an estimation equation that is derived based on a fact that the top layer thickness h bears linear relationship with an x coordinate of the point of inflection.

30. The soft tissue elasticity distribution measurement device according to claim 14,
wherein the computer determines the top layer thickness h by using an estimation equation that is derived based on a fact that the top layer thickness h bears linear relationship with an x coordinate of the point of inflection.

31. The soft tissue elasticity distribution measurement device according to claim 28,
wherein the computer determines the top layer thickness h by using an estimation equation that is derived based on a fact that the top layer thickness h bears linear relationship with an x coordinate of the point of inflection.

32. The soft tissue elasticity distribution measurement device according to claim 13,
wherein the shape of the aspiration aperture is isosceles triangular, and
the deformation amount measurement portion measures the aspiration deformation amounts of the soft tissue along the virtual line, which is coincident with an axis of symmetry of the aspiration aperture.

33. The soft tissue elasticity distribution measurement device according to claim 14,
wherein the shape of the aspiration aperture is isosceles triangular, and
the deformation amount measurement portion measures the aspiration deformation amounts of the soft tissue along the virtual line, which is coincident with an axis of symmetry of the aspiration aperture.

34. The soft tissue elasticity distribution measurement device according to claim 28,
wherein the shape of the aspiration aperture is isosceles triangular, and
the deformation amount measurement portion measures the aspiration deformation amounts of the soft tissue along the virtual line, which is coincident with an axis of symmetry of the aspiration aperture.

35. The soft tissue elasticity distribution measurement device according to claim 15,
wherein the shape of the aspiration aperture is isosceles triangular, and
the deformation amount measurement portion measures the aspiration deformation amounts of the soft tissue along the virtual line, which is coincident with an axis of symmetry of the aspiration aperture.

36. The soft tissue elasticity distribution measurement device according to claim 29,
wherein the shape of the aspiration aperture is isosceles triangular, and
the deformation amount measurement portion measures the aspiration deformation amounts of the soft tissue along the virtual line, which is coincident with an axis of symmetry of the aspiration aperture.

37. The soft tissue elasticity distribution measurement device according to claim 30,
wherein the shape of the aspiration aperture is isosceles triangular, and
the deformation amount measurement portion measures the aspiration deformation amounts of the soft tissue along the virtual line, which is coincident with an axis of symmetry of the aspiration aperture.

38. The soft tissue elasticity distribution measurement device according to claim 31,
wherein the shape of the aspiration aperture is isosceles triangular, and
the deformation amount measurement portion measures the aspiration deformation amounts of the soft tissue along the virtual line, which is coincident with an axis of symmetry of the aspiration aperture.
